(19) United States
(12) Patent Application Publication
(10) Pub. No.: US 2010/0285111 A1
(43) Pub. Date: Nov. 11, 2010

(54) SELF-ASSEMBLING MICELLE-LIKE NANOPARTICLES FOR SYSTEMIC GENE DELIVERY

(75) Inventors: Young Tag Ko, Brighton, MA (US); Amit Kale, Waltham, MA (US); Vladimir Torchilin, Charlestown, MA (US)

Correspondence Address: WILMERHALE/BOSTON, 60 STATE STREET, BOSTON, MA, 02109, US

(73) Assignee: Northeastern University, Boston, MA (US)

(21) Appl. No.: 12/741,778

(22) PCT Filed: Nov. 10, 2008
(86) PCT No.: PCT/US08/12660

§ 371 Date: May 6, 2010

Related U.S. Application Data
(60) Provisional application No. 61/002,626, filed on Nov. 9, 2007.

(51) Int. Cl. A61K 31/7088; A61K 9/14; C12N 15/63; C12N 15/64; C08G 73/00; A61K 9/127; A61P 35/00

(52) U.S. Cl. 424/450; 514/44 R; 424/497; 435/320.1; 435/440; 525/451; 525/54.2

(57) ABSTRACT

Nanoparticles containing nucleic acid and suitable for use as in vivo delivery agents for nucleic acids are provided. The nanoparticles use a covalent conjugate of a polycation such as polyethylenimine and phospholipids. The final DNA-containing nanoparticle has a vesicular structure with a polyplex core surrounded by a mixed lipid/PEG-lipid monolayer envelope and offers simple preparation, high loading capacity, and in vivo stability. The nanoparticles have good in vivo stability and a prolonged blood circulation time and can effectively deliver a gene to a biological target such as a tumor.

### CROSS REFERENCE TO RELATED APPLICATIONS

This application claims the priority of U.S. Provisional Application No. 61/002,626 filed Nov. 9, 2007 entitled, NANOPARTICLES FOR GENE DELIVERY, the whole of which is hereby incorporated by reference herein.

### STATEMENT REGARDING FEDERALLY SPONSORED RESEARCH OR DEVELOPMENT

The research leading to this invention was carried out with United States Government support provided under a grant from the National Institute of Health, Grant No. RO1 HL55519. Therefore, the U.S. Government has certain rights in this invention.

### BACKGROUND OF THE INVENTION

In vivo gene therapy depends on the delivery of DNA-based drugs, either in the form of oligonucleotides (antisense oligodeoxyribonucleotides (ODN), siRNA) or entire genes (plasmid DNA) to their cellular site of action. With few exceptions, where local administration may be feasible, progress towards broad clinical application of gene therapies requires the development of effective non-invasive delivery strategies. Non-viral systems are desirable as DNA vectors because these are safer, simpler to handle, and less expensive than viral vectors.

Among non-viral gene delivery systems, polymer-based polyplexes and lipid-based systems, either lipoplex or DNA entrapping liposome, have been explored but shown limited use for clinical application. This is mainly due to the lack of in vivo stability and thus inability to deliver the gene therapeutics to target sites at a therapeutic level. Tertiary lipopolyplex systems combining the polymer-based system and lipid-based system have been also explored. Among them, liposomal nanoparticles encapsulating PEI/DNA polyplex, such as bioPSL or pSPLP, have been proposed and tested for in vivo application with promising results. However, the combinatory systems involve complicated and time-consuming preparation steps and suffer from a low loading capacity despite in vivo stability and ability to reach the target sites with a long circulation time.

The cationic polymer polyethylenimine (polyethyleneimine, PEI) and its derivatives have been widely explored in gene delivery research [1-5]. PEI has the distinct advantage of the highest positive charge density among synthetic polycations, which enables effective condensation with DNA by electrostatic interaction. PEI is also endowed with an intrinsic mechanism mediating “endosomal escape” by the so called “proton sponge” mechanism [1, 2] and nuclear localization [6], which allows for high transfection efficiency. Available in a wide range of molecular weights from approximately 1 to 800 kDa and in linear or branched forms, low molecular weight PEI has been shown to be well tolerated, having low toxicity [7].

However, PEI, in the form of PEI/DNA complexes, has not shown significant therapeutic efficacy in vivo due to its rapid clearance from the circulation and accumulation within RES (reticuloendothelial system) sites. This is attributed mainly to the overall positive charge of the complexes. Although the positive charges of the complexes interact with negatively charged components of cell membranes and thus trigger cellular uptake of the complexes, they also cause interaction with blood components and opsonization leading to rapid clearance from the blood circulation. As a result, prior art PEI/DNA complexes are cleared from circulation in a few minutes and accumulate mainly in RES organs such as liver and spleen [8]. When injected systemically, these PEI/DNA complexes are also subject to DNA dissociation and aggregation in physiological environments [8]. These factors limit the in vivo application of known PEI/DNA complexes.

Several approaches have been tried to provide PEI/DNA complexes with improved in vivo stability [3, 5, 9]. As with other nanoparticulate systems [10], poly(ethlylene glycol) (PEG) has been used to confer in vivo stability to such complexes and prolong their circulation time. For this purpose, PEG has been covalently grafted to preformed PEI/DNA complexes [11], or PEG-grafted PEI has been used to form complexes with DNA [12]. Preformed PEI/DNA complexes were also coated with PEG using a copolymer of anionic peptide and PEG [13]. In combining PEI with liposome technology, lipid-grafted PEI such as cetylated PEI [14] and cholestery-PEI [15] have been used to prepare polycationic liposomes (PCL) loaded with DNA. Preformed PEI/DNA complexes have also been encapsulated in PEG-stabilized liposomes, resulting in the so-called “pre-condensed stable plasmid lipid particle” (pSPLP) [16]. However, other options are clearly needed to improve the success rate of in vivo gene therapy.

Among non-viral gene delivery systems, polymer-based polyplexes and lipid-based systems, either lipoplex or DNA entrapping liposome, have been explored but have shown limited use for clinical application. This is mainly due to the lack of in vivo stability and thus inability to deliver the gene therapeutics to target sites at a therapeutic level. Tertiary lipopolyplex systems combining the polymer-based system and lipid-based system have been also explored. Among them, liposomal nanoparticles encapsulating PEI/DNA polyplex, such as bioPSL or pSPLP, have been proposed and tested for in vivo application with promising results. However, the combinatory systems involve complicated and time-consuming preparation steps and suffer from a low loading capacity despite in vivo stability and ability to reach the target sites with a long circulation time.

### BRIEF SUMMARY OF THE INVENTION

To fulfill this need, a novel micelle-like nanoparticle (MNP) loaded with nucleic acid, such as plasmid DNA or siRNA, and a novel approach to constructing the nanoparticle for gene delivery have been developed. A cationic polymer, such as polyethylenimine (PEI), is first conjugated to the distal end of a phospholipid alkyl or acyl chain, resulting in a phospholipid-polyethylenimine (PLPEI) conjugate. The PLPEI is then mixed with a nucleic acid, such as plasmid DNA, oligonucleotides (e.g., antisense oligonucleotides), RNA or a ribozyme, to form complexes having a size in the nanometer range with the structure of a PEI/nucleic acid (PEI/NA) core complex and a phospholipid monolayer envelope. Electrostatic interaction between cationic PEI moieties of PLPEI and anionic nucleic acid provides a driving force toward the formation of the nanoparticles. Phospholipid moieties of the PLPEI conjugate are aligned to monolayer by hydrophobic interaction. Unmodified (i.e., unconjugated) phospholipids such as POPC, cholesterol are added to the PLPEI/nucleic acid complexes to supplement the lipid monolayer around the PEI/nucleic acid core. PEG-PE is also added to provide steric stabilization to the nanoparticles. The unmodified lipids and PEG-PE are incorporated into the monolayer via hydrophobic interaction. The final construct is a sterically stabililized micelle-like nanoparticle having a PEI/NA polyplex core and lipid monolayer envelope.

In a preferred embodiment, the nanoparticle according to the invention is based on a combination of a covalent conjugate between phospholipid and polyethylenimine (PLPEI), PEG-PE and lipids. A phospholipid-polyethylenimine conjugate can self-assemble into monolayer-enveloped hard-core micelle-like nanoparticles in the presence of plasmid DNA along with unmodified lipids and PEG-PE, and the resulting nanoparticles have architecture and properties suitable for in vivo application.

Nanoparticles according to the invention, a novel construct for gene delivery, are non-toxic, long-circulating, and effective for the in vivo transfection of therapeutic nucleic acids to both RES sites and other organs. This invention combines polymer-based gene delivery systems with lipid-based gene delivery systems, resulting in a new approach for using a chemical conjugate of phospholipids and polymer. The conjugation of polyethylenimine (PEI) at the distal end of phospholipid alkyl chain leads to a new chemical entity, a phospholipid-polyethylenimine (PLPEI) conjugate. The PLPEI possesses two functional domains for i) DNA binding and ii) membrane-formation, attributed to PEI and PL moieties, respectively. The PLPEI self-assembles, in the presence of DNA, into nanoparticles via electrostatic interaction of polycationic PEI with poly-anionic DNA. The self-assembly process is also facilitated by hydrophobic interaction between lipids moieties. The self-assembled nanoparticles possess a unique supramolecular structure in which the PEI/NA polyplex core and lipid monolayer envelope are connected by chemical bonds. The nanoparticle is different from, e.g., liposomal nanoparticles, where lipids form a bilayer instead of monolayer. The nanoparticle is also different from micelles, which assemble solely by hydrophobic interaction and are subject to “critical micelle concentration” limitation.

This invention provides advantages of a simple and reproducible one-step procedure in combination with a high loading capacity compared to other liposomal nanoparticle entrapping PEI/DNA polyplexes such as bioPSL and pSPLP. Nanoparticles according to the invention also provide for a high DNA loading capacity of around 25% (w/w), which is about 10-fold higher than values reported in the literature for other systems. As used herein, the term “DNA loading capacity” or “nucleic acid loading capacity” refers to the amount of DNA or other nucleic acid that can be incorporated into nanoparticles according to the invention.

### BRIEF DESCRIPTION OF THE DRAWINGS

Other features and advantages of the invention will be apparent from the following description of the preferred embodiments thereof and from the claims, taken in conjunction with the accompanying drawings.

FIG. 1 shows a schematic representation of the self-assembly process of micelle-like nanoparticles (MNP) with PEI/DNA core surrounded by the phospholipid monolayer. MNP form spontaneously in an aqueous media through the complexation of DNA with the phospholipid-polyethylenimine conjugate (PLPEI) followed by coating the complex with the lipid layer. The PEI moiety from PLPEI forms dense complexes with DNA resulting in a hydrophobic core, while the phospholipid moiety of PLPEI along with the unmodified lipids and PEG-PE forms the lipid monolayer that surrounds the PEI/DNA core. The lipid monolayer with incorporated PEG-PE provides also the in vivo stability.

FIGS. 2a-2b show an analysis of MNP formation. (FIG. 2a) Agarose gel electrophoresis of PLPEI/DNA complexes in comparison to PEI/DNA complexes at varying N/P ratios. No migration of the DNA into the gel indicates the complex formation. DNA was completely complexed by PLPEI at N/P≧6. The PLPEI showed complexation profile comparable to that of the unmodified PEI. (FIG. 2b) Freeze-fracture electron microscopy (ffTEM) analysis of MNP. MNP appear as well-developed spherical particles with an average diameter of 50 nm and a narrow size distribution. All particles display their shadow behind the structures, confirming micelle-like “hard-core” and “monolayer” structure. The bar indicates 50 nm.

FIGS. 3a-3b shows analysis of the stability of MNP. (FIG. 3a) Colloidal stability of NMP against salt-induced aggregation. Hydrodynamic diameters were monitored before and after adding salt (0.15 M NaCl). MNP remained stable while the PEI/DNA polyplexes showed rapid aggregation upon salt addition. Data represent mean±s.e.m. (n=3). (FIG. 3b) Protection of DNA loaded in MNP from the enzymatic degradation. MNP loaded with DNA and PEI/DNA polyplexes were analyzed on a 0.8% precast agarose gel after the treatment with DNAase I. DNA in MNP was completely protected from enzymatic degradation. Lane 1, DNA; lane 2, DNA, DNase; lane 3, PEI/DNA,; lane 4, PEI/DNA, DNAase; lane 5, MNP; lane 6, MNP, DNAase; lane 7, 100 base-pair ladder.

FIG. 4 shows the cytotoxicity of MNP towards NIH/3T3 cells. The fibroblast NIH/3T3 cells were treated with DNA-loaded MNP or with PEI/DNA polyplexes at different PEI concentration. Relative cell viability was expressed as a percentage of control cells treated with the medium. In contrast to PEI/DNA polyplexes, MNP showed no cytotoxicity after 24 hrs incubation following 4 hrs of treatments.

FIGS. 5a-5b shows the in vivo behavior of DNA-loaded MNP and PEI/DNA polyplexes in mice: (a) blood concentration-time curve (notice the logarithm scale), and (b) organ accumulation of DNA following the i.v. administration of the formulations carrying ¹¹¹In-labeled DNA. Blood was collected at different time points after the injection, and major organs were collected after the last blood sampling. Radioactivity of the blood and organ samples was measured by the gamma counter and expressed as a percentage of injected dose per ml blood or g tissue (% ID/ml or % ID/g). MNP showed a prolonged blood circulation and reduced RES uptake compared to PEI/DNA polyplexes. The p values were determined from the two-way analysis of variance (ANOVA) followed by Bonferroni post-hoc test.

FIGS. 6a-6b shows the results of in vivo transfection with pGFP-loaded MNP in a mouse xenograft model. The mice bearing LLC tumors were intravenously injected with MNP loaded with pGFP. At 48 hours post-injection, GFP expression in tumors was accessed. The fluorescence microscopy of frozen tumor sections from in vivo grown-LLC tumors is shown. (a) Tumor section from a non-treated animal (background pattern); (b) Tumor section from the animal injected with MNP loaded with pGFP. Intravenous injection of pGFP-loaded MNP led to bright fluorescence in a distal tumor. GFP expression in tumor tissues from the animals injected with PEI/DNA polyplexes was not accessed due to immediate death of the animals following injections with plains polyplexes at the same DNA content (n=3).

### DETAILED DESCRIPTION OF THE INVENTION

The inventors have developed a new gene delivery vector suitable for systemic application. The vector can be constructed using a chemical conjugate of phospholipids and a polycation such as polyethylenimine (PLPEI) at the distal end of the alkyl chain. The electrostatic interaction of polycationic PEI moieties with DNA drives the formation of dense PEI/DNA polyplex cores while the amphiphilic phospholipid moieties, together with optionally added free unmodified phospholipids and PEG-grafted phospholipids (e.g., PEG-PE) form a lipid monolayer envelope around the polyplex cores and lead to the formation of DNA-loaded micelle-like nanoparticles (MNP) stabilized by a steric barrier of PEG chains and a membrane-like barrier of a lipid monolayer envelope.

In contrast to dramatic successes with sterically stabilized liposomes[20], the steric stabilization of polyplexes by polyethylene glycol (PEG) has not successfully provided both circulatory longevity and in vivo stability [8]. Significant improvement has been achieved with the present invention in which steric stabilization is combined with the “lateral stabilization” by crosslinking the surface of the polyplexes [9]. This demonstrates that steric stabilization plays only a limited role in the in vivo stability of polyplexes, and that additional stabilization mechanisms are necessary to confer added in vivo stability to polyplexes.

The additional stabilization can be achieved by enveloping the polyplexes within a lipid barrier since the lipid barrier is impermeable to salts and thus prevents the polyplex cores from salt-induced instability. In vivo behavior of such systems is governed by the lipid barrier, while the polyplex core is shielded from the biological environment in the blood circulation. Steric stabilization of the lipid barrier provides the loaded polyplexes with a prolonged circulation time and makes it possible to deliver the polyplexes to target organs other than RES sites via the EPR mechanism. Furthermore, upon the cellular uptake, PEI is still expected to exert its favorable functions, such as the endosomolytic activity and its protection from cytoplasmic nucleases to improve an intracellular pharmacokinetics of the DNA molecules.

Micelle-like nanoparticles are additionally stabilized by the presence of the envelope of the lipid monolayer, which forms by a self-assembly process driven by the hydrophobic interactions between the lipid moieties of PLPEI together with free lipids and PEG-lipids. The strong resistance of the MNP against the salt-induced aggregation and enzymatic digestion confirms the presence of such a lipid monolayer barrier. The high salts in physiological conditions provide one of the mechanisms responsible for the poor in vivo stability of PEI/DNA polyplexes [8]. These polyplexes are formed by strong electrostatic interaction between polycationic PEI and polyanionic DNA molecules and colloidally stabilized by electrostatic repulsion between the particles. Under the physiological conditions, however, an increased salt concentration triggers the aggregation of polyplex particles as a result of screening of the electrostatic repulsion forces between the polyplex particles along with concurrent dissociation of the polyplex particles due to screening of attractive electrostatic interaction between polycations and polyanionic DNA [21]. Although steric stabilization by PEG chains resulted in a decreased sensitivity of the polyplexes of PEG-grafted PEI to the salt-induced aggregation, the moderate stability of the polyplexes suggests that steric stabilization alone plays a limited role and additional stabilization mechanisms are required to prevent the aggregation of the polyplexes [12, 22-24]. The existence of the salt-impermeable lipid barrier contributes to the observed stability of the MNP in high salt conditions. The lipid monolayer barrier, as with liposomes, blocks the access of salts from the outer environment to the polyplex cores and thus provides protection against the salt-induced aggregation to the otherwise unstable polyplexes. The moderate aggregation with the intermediate PLPEI/DNA complexes without free lipids indicates that the phospholipid moieties of the PLPEI conjugates alone might not provide as complete a lipid barrier as when the conjugated phospholipids are supplemented with non-conjugated lipids.

The amount of PEG-lipid such as PEG-PE was chosen to facilitate the incorporation of free lipids into the preformed complexes and also to provide steric stabilization of the final construct. Considering that mixtures of PEG-PE with phospholipids evolve from a micelle phase to lamellar phase as the PEG-PE content in the mixture increases with the onset of micelle formation at ˜5 mol % [25, 26], the aqueous suspension of the free lipid mixture with a 10 mol % PEG-PE concentration favors the micelle phase transition to the lamellar phase. Upon the incubation with the preformed PLPEI/DNA complexes, the PEG-PE content of total lipids comprising the free and the conjugated lipids decreases to 4.3 mol %, at which a lamellar phase is favored. It has also been shown that PEG-PE molecules in a micelle phase spontaneously incorporate in the surface of preformed phospholipid vesicles by so called “micelle transfer” [27]. Free lipids can be expected to interact with hydrophobic lipid domains of PLPEI/DNA polyplexes, leading to spontaneous incorporation of free lipids into the lipid layer of the preformed complexes following dissociation into monomers and thus, along with the phospholipids moieties from PLPEI conjugates, form a lipid monolayer envelope surrounding the polyplex core. The final construct is a sterically stabilized micelle-like hard-core particle with a PEI/DNA polyplex core and lipid monolayer envelope.

A similar hydrophobic interaction was proposed as a plausible stabilizing mechanism of Pluronic P123-grafted PEI/DNA polyplex systems [28, 29], in which the amphiphilic Pluronic P123 chains of Pluronic P123-grafted PEI form a micelle-like structure around the polyplex core and unmodified Pluronic P123 was incorporated into the polyplexes by the hydrophobic interaction with Pluronic P123-grafted PEI conjugates and thus filled in to optimize the stability of the micelle-like structure.

Micelle-like nanoparticles, in a sense, resemble so called “liposome-entrapped polycation-condensed DNA particle” (LPD II) entrapping polylysine/DNA within folate-targeted anionic liposomes [30], or ‘artificial virus-like particles’ prepared by entrapping PEI/DNA polyplexes within preformed anionic liposomes [31-33], or “pre-condensed stable plasmid lipid particles” (pSPLP)[16] constructed by encapsulating PEI/DNA polyplexes within a lipid bilayer stabilized by an external PEG layer. In particular, pSPLP demonstrate advantages of encapsulating polyplexes within stabilized liposomes, i.e. the effective systemic delivery of PEI/DNA polyplexes to tumors due to the prolonged circulation time and improved transfection potency due to the endosomolytic activity of PEI. However, the preparation of pSPLP involves a potentially damaging incubation of preformed polyplexes with lipids in ethanol (organic solvent) and thus requires multiple steps of concentration and dialysis.

Micelle-like nanoparticles offer the advantages of combining polyplexes with a sterically stabilized lipid membrane, albeit a monolayer in this case. The PLPEI conjugate enables a process of self-assembly of DNA-loaded MNP by simultaneous DNA condensation and lipid membrane formation. Compared to liposome-encapsulated DNA-PEI complexes, MNP provide a more convenient one-step DNA loading with 100% efficiency and also allow a loading capacity (up to 530 μg DNA/μmole total lipids, or 30% of total particle mass as nucleic acid), higher than any method of DNA encapsulation into a liposomal formulation [34].

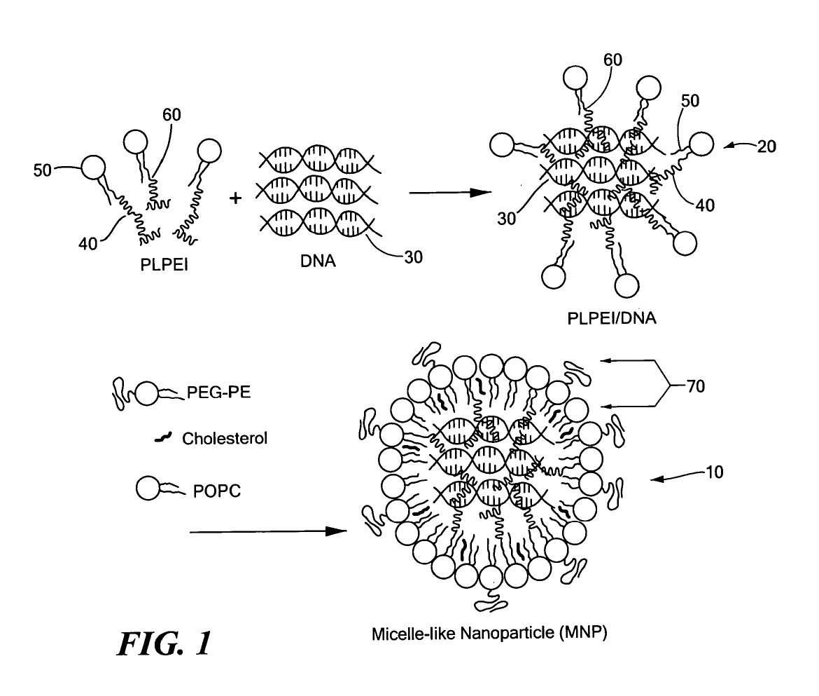

A micelle-like nanoparticle 10 according to the present invention contains a core complex encapsulated by a lipid monolayer (see FIG. 1). The core complex 20 contains one or more nucleic acid molecules 30 that are electrostatically bound to one or more molecules of a cationic polymer 40, such as PEI. The cationic polymer is covalently conjugated to a lipid molecule 50 that resides in the encapsulating lipid monolayer. On the one hand, the cationic polymer serves to bind and package the nucleic acid to form the core complex of the nanoparticle. On the other hand, the cationic polymer provides a covalent linkage 60 to the hydrophobic portion of a lipid molecule, preferably a phospholipid, thereby mediating the encapsulation of the core complex with a monolayer of lipid 70 to promote stability and the ability to fuse with cell membranes.

Micelle-like nanoparticles can have an average diameter in the range from about 10 nm to about 1000 nm. Preferably they have an average diameter in the range from about 10 nm to about 500 nm, more preferably from about 10 nm to about 200 nm, and even more preferably from about 40 nm to about 100 nm or about 50 nm to about 70 nm. The size of MNP is compatible with their ability to enter cells and transfer their nucleic acid content into the cytoplasm of the cell.

The cationic polymer can be any synthetic or natural polymer bearing at least two positive charges per molecule and having sufficient charge density and molecular size so as to bind to nucleic acid under physiological conditions (i.e., pH and salt conditions encountered within the body or within cells). Suitable cationic polymers include, for example, polyethylene imine, polyornithine, polyarginine, polylysine, polyallylamine, and aminodextran. Cationic polymers can be either linear or branched, can be either homopolymers or copolymers, and when containing amino acids can have either L or D configuration, and can have any mixture of these features. Preferably, the cationic polymer molecule is sufficiently flexible to allow it to form a compact complex with one or more nucleic acid molecules.

A lipid molecule that is conjugated to a cationic polymer is herein referred to as a “first lipid”, “first phospholipid”, “conjugated lipid” or “conjugated phospholipid”. Suitable lipids include any natural or synthetic amphipathic lipid (also referred to as amphiphilic lipid) that can stably form or incorporate into lipid monolayers or bilayers in combination with other amphipathic lipids. The hydrophobic moiety of the lipid is in contact with the hydrophobic region of a monolayer or bilayer and its polar head group moiety oriented toward the aqueous phase at the exterior, polar surface of a monolayer or bilayer, and in this case towards the exterior surface of the nanoparticle. Hydrophilic characteristics of amphipathic lipids derive from the presence of polar or charged groups such as carbohydrates, phosphate, carboxylic, sulfate, amino, sulfhydryl, nitro, hydroxy and similar groups. The hydrophobic portion of an amphipathic lipid can be conferred by the inclusion of non-polar groups including long chain saturated and unsaturated aliphatic hydrocarbon groups and such groups substituted by one or more aromatic, cycloaliphatic or heterocyclic group(s). Examples of amphipathic lipids include, but are not limited to, natural or synthetic phospholipids, glycolipids, aminolipids, sphingolipids, long chain fatty acids, and sterols. Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine and dilinoleoylphosphatidylcholine. Other compounds lacking in phosphorus, such as sphingolipids, glycosphingolipids, diacylglycerols, and β-acyloxyacids also can be used as amphipathic lipids.

In certain embodiments, a nanoparticle according to the invention contains additional lipids that are not conjugated to a cationic polymer (“non-conjugated lipid” or “non-conjugated phospholipid”). These additional, non-conjugated lipids serve to stabilize and complete the encapsulating lipid monolayer, and also can serve as attachment points for stabilizing moieties (e.g., PEG) or targeting moieties. Non-conjugated lipids can be any of the amphipathic lipids described above, such as phospholipids, and also can include other lipids such as triglycerides and sterols (e.g., cholesterol). At least one of the conjugated and non-conjugated lipids in a nanoparticle should be a bilayer forming lipid such as, for example, a phospholipid. In a preferred embodiment, the lipid monolayer of the nanoparticle contains a first portion of conjugated lipid, a second portion of non-conjugated lipid, and a third portion of cholesterol. The relative amounts of each portion can vary, but are preferably in the range of about 10 to 70% by mole fraction of the monolayer lipids for each of the conjugated and non-conjugated lipids, and in the range of about 1 to 30%, or about 5% to 20%, by mole fraction of the monolayer lipids for cholesterol. For example, in one embodiment the lipid monolayer contains conjugated lipid, non-conjugated lipid, and cholesterol at a ratio of 4:3:3 respectively.

The lipid monolayer of the MNP can contain a variety of additional molecular constituents whose purpose can be, for example, to stabilize or label the particle or to endow it with a targeting function. Such constituents include peptides, proteins, detergents, lipid-derivatives, and especially PEG-lipid derivatives such as PEG coupled to dialkyloxypropyls, diacylglycerols, phosphatidylethanolamines, and ceramides (see, e.g., U.S. Pat. No. 5,885,613, which is incorporated herein by reference). In certain embodiments, the nanoparticles are essentially detergent free. Where PEG-lipids are added to the monolayer, they are preferably present in an amount corresponding to about 0.5 to 20% by weight of the monolayer lipid, more preferably about 1 to 10%, and still more preferably about 2 to 5%. In a preferred embodiment, the lipid monolayer of the nanoparticle contains conjugated lipid, non-conjugated lipid, cholesterol, and PEG-PE at a mole ratio of 4:3:3:0.3 respectively.

A lipid derivative that is useful for attaching peptides or proteins to the nanoparticle is p-nitrophenylcarbonyl PEG-PE (pNP-PEG-PE). Free amino groups, e.g., on an antibody or other protein molecule, can react with the pNP group to covalently attach targeting moieties to the nanoparticles. See, e.g., Liposomes: A Practical Approach, V. P. Torchelin and V. Weissig, Oxford University Press, 2003, which is hereby incorporated by reference.

The central core complex of the nanoparticle contains, in addition to the cationic polymer, one or more nucleic acid molecules. These nucleic acids are generally intended for transfer to living cells or tissues where they are expected to exert a biological action. The term “nucleic acid” refers to deoxyribonucleotides or ribonucleotides and polymers thereof (DNA or RNA) in single- or double-stranded form. The term encompasses nucleic acids containing known nucleotide analogs or modified backbone residues or linkages, which are synthetic or naturally occurring. Examples of such analogs include, without limitation, phosphorothioates, phosphoramidates, methyl phosphonates, chiral-methyl phosphonates, 2-O-methyl ribonucleotides, and peptide-nucleic acids (PNAs). DNA can be in the form of antisense, plasmid DNA, parts of a plasmid DNA, pre-condensed DNA, a product of a polymerase chain reaction (PCR), vectors (Pl, PAC, BAC, YAC, artificial chromosomes), expression cassettes, chimeric sequences, chromosomal DNA, or derivatives of any of these. The term nucleic acid is used interchangeably with the terms gene, cDNA, mRNA encoded by a gene, and an interfering RNA molecule. The term “gene” refers to a nucleic acid (e.g., DNA or RNA) sequence that comprises partial or full length coding sequences necessary for the production of a polypeptide or a polypeptide precursor.

The term “RNAi” refers to double-stranded RNA that is capable of reducing or inhibiting expression of a target gene by mediating the degradation of mRNAs which are complementary to the sequence of the interfering RNA, when the interfering RNA is in the same cell as the target gene. RNAi thus refers to the double stranded RNA formed by two complementary strands or by a single, self-complementary strand. RNAi typically has substantial or complete identity to the target gene. The sequence of the interfering RNA can correspond to the full length target gene, or a subsequence thereof. RNAi includes small-interfering RNA or “siRNA”. siRNA contain about 15-60, 15-50, 15-50, or 15-40 base pairs in length, more typically about, 15-30, 15-25 or 19-25 base pairs in length, and are preferably about 20-24 or about 21-22 or 21-23 base pairs in length. siRNA duplexes may comprise 3′ overhangs of about 1 to 4 nucleotides, preferably of about 2 to 3 nucleotides and also may contain 5′ phosphate termini. siRNA can be chemically synthesized or can be encoded by a plasmid. siRNA can also be generated by cleavage of longer dsRNA. Preferably, dsRNA are at least about 100, 200, 300, 400 or 500 nucleotides in length. A dsRNA may be as long as 1000, 1500, 2000, 5000 nucleotides in length, or longer. The dsRNA can encode for an entire gene transcript or a partial gene transcript.

The ratio of cationic polymer to nucleic acid molecules for packaging into nanoparticles of the invention should be adjusted to ensure that all of the nucleic acid is complexed. A gel electrophoretic method for achieving this is described in the examples below. Generally, a ratio of amine to phosphate (N/P) in the range of about 1 to 20 is appropriate. A ratio of about 10 is preferred. The amount of nucleic acid that can be loaded into an individual MNP can vary over a broad range. The nucleic acid content of the completed MNP can be up to 40% by weight, which is much higher than is possible with previously described nucleic acid-containing nanoparticles. For some techniques, only a very small amount of nucleic acid, or even no nucleic acid (e.g., control particles) may be required; in such cases a portion of the cationic polymer can be complexed with an anionic polymer (e.g., carboxymethyl cellulose) in order to form a stable core. The proportion of charged groups in the cationic polymer and the nucleic acid can vary depending on the pH of the solution in which they are combined. The polymer can be designed such that a desired proportion of the ionizable groups is charged for combination with nucleic acid. For example, at least about 10% of the groups are charged (e.g., positively charged) in some embodiments, whereas in preferred embodiments about 50 to 100% of the groups on the polymer are charged during formation and in the completed core complex.

Generally, it is desired to deliver the MNPs of the present invention to down regulate or silence the expression of a gene product of interest. Alternatively, a therapeutic gene can be delivered to certain cells in order to replace a defective gene, to increase the expression of a gene product, or to regulate the expression of other genes. Many gene products suitable as targets of the MNPs of the invention are known to those of skill in the art. These include, but are not limited to, genes associated with viral infection and survival, genes associated with metabolic diseases and disorders, genes associated with tumorigenesis and cell transformation, angiogenic genes, immunomodulator genes such as those associated with inflammatory and autoimmune responses, ligand receptor genes, and genes associated with neurodegenerative disorders. Any suitable target can be selected by the user, who can routinely select an appropriate RNAi or therapeutic gene sequence.

The invention further provides a non-viral vector that contains a nanoparticle as described above. In addition to including a core complex having a nucleic acid-cationic polymer complex, and an encapsulating lipid monolayer containing a first phospholipid that is conjugated at its distal (hydrophobic) end to the cationic polymer, the vector is suitable for transferring the nucleic acid from the core complex into a cell. This can be accomplished by any of a variety of mechanisms, such as, for example, the inclusion of membrane fusion-promoting lipids or proteins in the lipid monolayer of the vector, or the inclusion of one or more targeting agents, such as a ligand or antibody, that binds to a receptor found on the surface of the target cells. Furthermore, the vector can include nucleic acid sequences designed to promote or regulate the expression or genomic incorporation of other nucleic acid sequences of the vector.

In order to deliver the nanoparticles and non-viral vectors according to the invention to the appropriate cells for transfer of their nucleic acid contents, a targeting agent or targeting moiety can be added to the surface of the nanoparticles during their formation. This is readily accomplished by including the targeting agent among the non-conjugated lipids, which can be conveniently accomplished using a lipid derivative of the targeting moiety. For example, many targeting agents are peptides or proteins, which can be conjugated to a lipid via available chemical side chains (e.g., amino groups on the targeting agent reacted with pNP-PEG-PE). Suitable targeting agents are known in the art, and include, but are not limited to, naturally occurring or engineered antibodies or antigen binding fragments thereof, domain or single chain antibodies, ligands for cell surface receptors, biotin, and the like.

Another aspect of the present invention is a method of making a micelle-like nanoparticle containing a core complex encapsulated by a lipid monolayer. One or more nucleic acid molecules are contacted with a cationic polymer-lipid conjugate as described above under conditions suitable to form a complex that will form the core of the nanoparticle. The negatively charged nucleic acid electrostatically binds to the cationic polymer portion of the conjugate to form a stable core complex. The core complex is then supplemented with one or more non-conjugated lipids to form a lipid monolayer that encapsulates the core complex.

Yet another aspect of the invention is a method of transfecting a cell with a micelle-like nanoparticle. The cell is contacted with the non-viral vector described above under conditions suitable for transfer of a nucleic acid molecule of the vector into the cell.

The nanoparticles and non-viral vectors of the present invention can be administered either alone or as a pharmaceutical composition containing the nanoparticles together with a pharmaceutical carrier such as physiological saline or phosphate buffer, selected in accordance with the route of administration and standard pharmaceutical practice. The pharmaceutical carrier is generally added following particle formation. The concentration of particles in the pharmaceutical formulations can vary widely, i.e., from less than about 0.05%, or about 2.5%, to as much as 10 to 30% by weight.

Pharmaceutical compositions of the present invention may be sterilized by conventional, well known sterilization techniques. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, and calcium chloride. Additionally, the particle suspension may include lipid-protective agents which protect lipids against free-radical and lipid-peroxidative damage on storage. Lipophilic free-radical quenchers, such as alpha-tocopherol, can be used for example.

The nanoparticles and non-viral vectors of the present invention can be used to introduce nucleic acids into cells, e.g., to treat or prevent a disease or disorder associated with expression of a target gene. Accordingly, the present invention also provides methods for introducing a nucleic acid (e.g., an RNAi or a therapeutic gene) into a cell. In a method of treating a subject having a disease or medical condition, or preventing a disease or medical condition in a subject, a non-viral vector according to the present invention is contacted with one or more cells either in vivo or in vitro. The cells can be cells of the subject or cells provided by a donor. As a result of contacting the cells with the vector, one or more nucleic acid molecules of the vector are transferred into cells of the subject, whereby the disease or medical condition is treated or prevented. In some embodiments where the cells are contacted with the vector in vitro, the cells then can be administered to the subject as part of treatment or prevention. Suitable micelle-like nanoparticles are formed as described above. The particles are then contacted with the appropriate target cells for a period of time sufficient for delivery of nucleic acid to occur. The nanoparticles of the present invention can be adsorbed to almost any cell type with which they are mixed or contacted. Once adsorbed, the particles can either be internalized by endocytosis, exchange with lipids at cell surface membranes, or fuse with the target cells, whereupon transfer or incorporation of nucleic acid from the particle to the cell can take place. Among the cell types most often targeted for intracellular delivery of a nucleic acid are neoplastic cells (tumor cells). Other cells that can be targeted include hematopoietic precursor cells or stem cells, fibroblasts, keratinocytes, hepatocytes, endothelial cells, skeletal and smooth muscle cells, osteoblasts, neurons, quiescent lymphocytes, terminally differentiated cells, lymphoid cells, epithelial cells, bone cells, and the like.

For in vitro applications, the delivery of nucleic acids by nanoparticles according to the present invention can be to any cell grown in culture, whether of plant or animal origin, vertebrate or invertebrate, and from any tissue. Contact between the cells and the nanoparticles, when carried out in vitro, takes place in a biologically compatible medium. The concentration of particles can vary depending on the particular application. Treatment of cells in vitro with the nanoparticles is generally carried out at physiological temperatures (about 37° C.) for periods of time of from about 1 to 48 hours, preferably about 2 to 4 hours.

A method of suppressing the expression of a gene in a cell is provided. The method includes contacting the cell with a micelle-like nanoparticle whose core complex contains siRNA or RNAi, or a nucleic acid that generates RNAi or siRNA within a target cell. The siRNA or RNAi is transferred into the cell and suppresses the expression of a gene of interest, for which the siRNA or RNAi sequence is specifically designed according to known methods.

In some embodiments, the nanoparticles can be used for in vivo delivery of nucleic acids such as siRNA or therapeutic genes to animals, such as canines, felines, equines, bovines, ovines, caprines, rodents, or primates, including humans. In vivo delivery can be local, i.e., directly to the site of interest, or systemic. Systemic delivery for in vivo gene therapy, i.e., delivery of a therapeutic nucleic acid to a distal target cell via body systems such as the circulation, has been achieved using nucleic acid-lipid particles such as those disclosed in published PCT Patent Application WO 96/40964, U.S. Pat. Nos. 5,705,385, 5,976,567, 5,981,501, and 6,410,328, all of which are incorporated herein by reference.

The present invention also provides micelle-like nanoparticles in kit form. A kit will typically include a container and one or more compositions of the present invention, with instructions for their use and administration. In some embodiments, the nanoparticles will have a targeting moiety already attached to their surface, while in other embodiments the kit will include nanoparticles that can be reacted with the user's choice of targeting moiety. Methods of attaching targeting moieties (e.g., antibodies, proteins) to lipids in the encapsulating monolayer are known to those of skill in the art, and the kit can supply instructions for such methods.

Another aspect of the invention is a chemical conjugate that contains a cationic polymer covalently bound to a distal end of a lipid acyl or alkyl chain. Such chemical conjugates are used in preparing MNP, and also have other uses in preparing micelle-, monolayer-, or bilayer-containing structures for use in commercial products such as drugs, cosmetics, foods, diagnostic tools, medical devices and their coatings, and biosensors. The chemical conjugate includes one or more of the polymeric cations described earlier, such as polyethyleneimine, which is chemically conjugated to the distal, hydrophobic portion of an amphipathic lipid molecule. The chemical conjugation is by a covalent bond, and in some embodiments this bond is cleavable under certain conditions, such as acidic pH or the action of an enzyme. For example, the conjugate can be formed by reacting 1-palmitoyl-2-azelaoyl-sn-glycero-3-phosphocholine with polyethyleneimine. The chemical conjugate can be bound to one or more nucleic acid molecules to form a nucleic acid-polycation-lipid complex.

The following examples are presented to illustrate the advantages of the present invention and to assist one of ordinary skill in making and using the same. These examples are not intended in any way otherwise to limit the scope of the disclosure.

### Materials and Methods

### Materials

All materials were purchased from Sigma-Aldrich unless otherwise stated. Plasmid DNA (pDNA) encoding Green Fluorescence Protein (GFP) was purchased at a final concentration of 1 μg/μl from Elim Biopharmaceuticals (Hayward, Calif.). Rhodamine labeled pGFP (pGeneGrip Rhodamine/GFP) was purchased from Genlantis (San Diego, Calif.). When necessary, the DNA was radioactively labeled with ¹¹¹In (PerkinElmer Life and Analytical Sciences, MA) to obtain 0.1 μCi/μg DNA according to methods described previously[17]. The concentration and purity were checked by 0.8% agarose gel electrophoresis. 1-Palmitoyl-2-oleoyl-sn-glycero-3-phosphocholine (POPC), 1,2-disrearoyl-sn-glycero-3-phosphoethanolamine-N-[methoxy(polyethylene glycol)-2000] (PEG-PE), cholesterol and oxidized phospholipid, 1-palmitoyl-2-azelaoyl-sn-glycero-3-phosphocholine (AzPC Ester) were purchased from Avanti Polar Lipids (Alabaster, Ala.). Branched PEI (bPEI) with MW 1.8 kDa was purchased from Polysciences, Inc. (Warrington, Pa.) and dissolved in water to a final concentration of 1.0 μg/μl.

### Synthesis of Phospholipid-Polyethylenimine Conjugate (PLPEI)

Twelve miligrams of the branched PEI (7 μmole) were dissolved in 0.5 ml of chloroform and mixed with five miligrams of the oxidized PC (AzPC Ester, 7 μmole) dissolved in 1 ml of chloroform. Assuming that bPEI has 1:2:1 molar ratio of primary:secondary:tertiary amines, the reaction mixture corresponds to an acid-to-primary amine molar ratio of 1:10, i.e. contains an excess reactive amines. A half milligram of carbonyldiimidazole (CDI, 3 μmole) was added to the above solution for the activation of acid by forming an imidazolide derivative. The reaction mixture was incubated with 10 μl of TEA (triethylamine) at room temperature for 24 hrs with stirring. The chloroform was then removed under a stream of nitrogen gas and the residue was suspended with 2 ml of dH₂O. The products were purified by dialysis against dH₂O (MWCO 2,000 Da), lyophilized and their structure was confirmed by the ¹H-NMR (in CDCl₃, 300 MHz). The extent of conjugation was determined to be 1:1 molar ratio of PEI to lipid from the ratio of ethylene (—CH₂CH₂—) signal (2.4-2.8 ppm) of the PEI main chain to methyl (—CH₃) signal of the phospholipids head (3.4 ppm) on the NMR spectrum (δ 0.9:2.7 H, δ 1.3:17.6 H, δ 1.6:5.4 H, δ 2.4-2.8:96.0 H, δ 3.3:12.8 H, δ 3.6:1.58 H, δ 4.0-4.6:5.43 H). The PLPEI conjugate was dissolved in water to a concentration of 1.5 μg/μl (1.0 μg/μl as of PEI).

### Complexation of Plasmid DNA With PLPEI

Constant amounts of plasmid DNA (100 μg) and varying amounts of PLPEI were separately diluted in HBG (10 mM HEPES, 5% d-Glucose, pH 7.4) to the final volume of 250 μl. The PLPEI solution was then transferred to the DNA solution by fast addition and vortexed. The resulting polyplexes were analyzed by agarose gel electrophoresis using the E-Gel elctrophoresis system (Invitrogen Life Technologies). A precast 0.8% E-Gel cartridge was pre-run for 2 min at 60 V and 500 mA followed by loading of 1 μg of pDNA. The desired amine/phosphate (N/P) ratio was calculated assuming that 43.1 g/mol corresponds to each repeating unit of PEI containing one amine, and 330 g/mol corresponds to each repeating unit of DNA containing one phosphate.

### Preparation of Micelle-Like Nanoparticles Encapsulating Plasmid DNA (MNP)

The MNP were constructed with PLPEI:POPC:Cholesterol:PEG-PE (4:3:3:0.3, mol/mol) and pDNA. First, PLPEI (130 μg as PEI) and plasmid DNA (100 μg) corresponding to N/P ratio of 10 were separately diluted in HBG to final volume of 250 μl. The PLPEI solution was transferred to the DNA solution by fast addition and vortexed. Dry lipid film was separately prepared from the mixture of POPC, cholesterol, and PEG-PE (42 μg, 21 μg, 15 μg, 3:3:0.3 mol/mol) and hydrated with 500 μl of HBG. The lipid suspension was incubated with the preformed PLPEI/DNA complexes for 24 hours at room temperature. Alternatively, the PLPEI/DNA complex was added directly to the lipid film. The resulting suspension of MNP was stored at 4° C. until use.

### Size and Zeta Potential

The MNP were diluted in HBG to obtain an optimal scattering intensity. Hydrodynamic diameter and zeta potential were measured by the quasi-electric light scattering (QELS) using a Zeta Plus Particle Analyzer (Brookhaven Instruments Corp, Santa Barbara, Calif.). Scattered light was detected at 23° C. at an angle of 90°. A viscosity value of 0.933 mPa and a refractive index of 1.333 were used for the data analysis. The instrument was routinely calibrated using a latex microsphere suspension (0.09 μm, 0.26 μm; Duke Scientific Corp, Palo Alto, Calif., USA).

### Freeze-Fracture Electron Microscopy

The MNP were quenched using the sandwich technique and liquid nitrogen-cooled propane. At a cooling rate of 10,000 K/sec to avoid ice crystal formation and other artifacts of the cryofixation process. The fracturing process was carried out in JEOL JED-9000 freeze-etching equipment, and the exposed fracture planes were shadowed with platinum for 30 sec at an angle of 25-35° and with carbon for 35 sec [2 kV, 60-70 mA, 1×10⁻⁵ torr (1 torr=133 Pa)]. The replicas were cleaned with fuming HNO₃ for 24-36 h followed by repeated agitation with fresh chloroform/methanol [1:1 (vol/vol)] at least five times and examined with a JEOL 100 CX electron microscope.

### Stability Against Salt-Induced Aggregation

Colloidal stability of the MNP particles against the salt-induced aggregation was determined by monitoring the MNP size (hydrodynamic diameter). NaCl (5 M) was added to the MNP in HBG to a final concentration of 0.15 M while measuring the size as described above.

### Nuclease Resistance

Nuclease resistance of the DNA molecules in MNP particles was determined by treating the samples with 50 units of DNase I (Promega Corp., Madison, Wis.) for 30 min at 37° C. The reaction was terminated using EGTA and EDTA at a final concentration of 5 mM. The DNA molecules were dissociated using heparin (50 units/μg of DNA) at 37° C. for 30 min, and the products were analyzed on a 0.8% precast agarose gel.

### Cytotoxicity Assay

The fibroblast NIH/3T3 cells were grown in DMEM supplemented with 10% fetal bovine serum (FBS) in 96-well plates. The cells were treated by replacing the media with serum-free media (100 μl) containing a serial dilution of each formulation up to 100 μg/ml of PEI. After 4 hrs incubation, the cells were washed twice with PBS and returned to complete media (100 μl). After 24 hrs incubation, 20 μl of CellTiter 96 Aqueous One solution (Promega, Madison, Wis.) was added to each well and the plates were re-incubated for 2 hrs. The absorbance at 490 nm was measured for each well using a 96-well plate reader (Multiscan MCC/340, Fisher Scientific Co). Relative cell viability was calculated with cells treated only with the medium as a control.

### Pharmacokinetics and Biodistribution

Male balb/c mice (20-30 g) were maintained on anesthesia with ketamine/xylazine (1 mg/0.2 mg/animal) and catheterized with PE-10 in a retrograde direction via the right common carotid artery according to a protocol approved by the Institutional Animal Care and Use Committee at Northeastern University. The MNP loaded with ¹¹¹In-DNA (˜2 μCi ¹¹¹In, 20 μg DNA) were injected through a tail vein. Blood samples (30 μl) were taken through the catheter in the common carotid artery at 1, 2, 5, 10, 30, 60 min after the intravenous bolus injection. The sample volume was replaced with PBS containing heparin (10 U/ml). After the last blood sampling at 60 min. the animals were sacrificed by cervical dislocation and organ samples (lung, liver, spleen, kidney, muscle, and skin) were taken. Radioactivity of the blood and organ samples was measured by a γ-counter. The radioactivity was expressed as percentage of injected dose (% ID/g for organ, % ID/ml for blood). Organ distribution values were corrected for blood volume of the corresponding organs. Pharmacokinetic parameters were determined by fitting blood “concentration vs time” data to a biexponential equation (C(t)=A*e^{-αt}+B*e^{-βt}).

### In Vivo Gene Expression

Male C57BL/6 mice (Charles River Laboratories) were inoculated subcutaneously in the left flank with 1×10⁶ LLC tumor cells 14 days before treatment according to a protocol approved by the Institutional Animal Care and Use Committee at Northeastern University. MNP containing 40 μg pGFP in a 200 μl injection volume were administered by the tail vein injection. Noninjected mice with similar-sized tumors were used as negative controls. Anesthetized mice were sacrificed 48 hrs later by cervical dislocation, and excised tumors were immediately frozen in Tissue-Tek OCT 4583 compound (Sakura Finetek, CA) without fixation and 8 μm thick sections were prepared with a cryostat. GFP fluorescence was visualized with a fluorescence microscopy (Olympus BX51).

### Example I

### Preparation of Micelle-Like Nanoparticles (MNP)

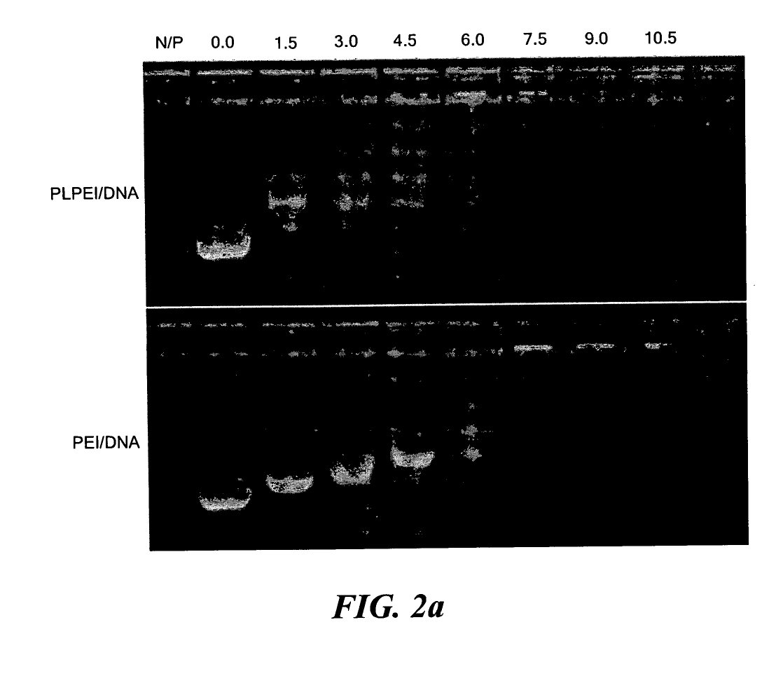

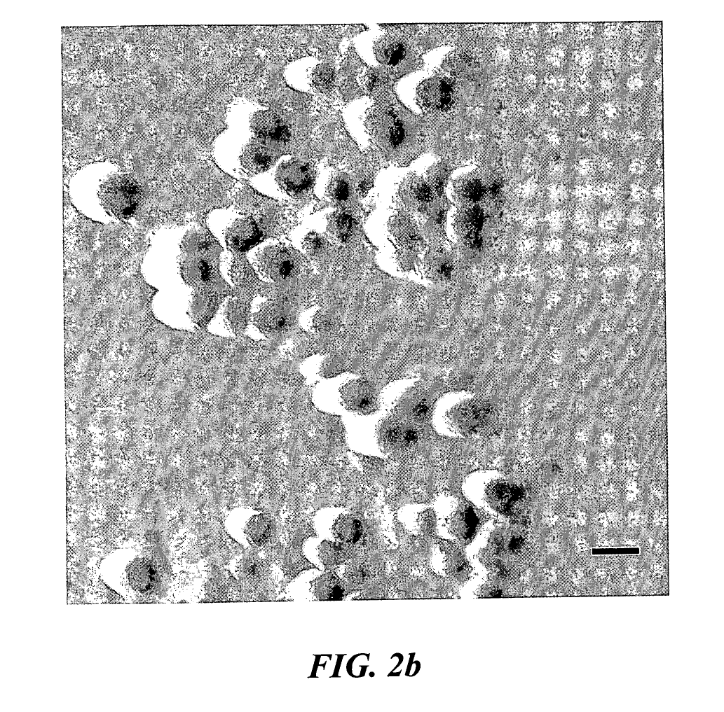

The micelle-like nanoparticles (MNP) were prepared by complexing plasmid DNA with PLPEI and then enveloping the preformed complexes with a lipid layer containing also PEG-phosphatidylethanolamine conjugate (PEG-PE) (FIG. 1). As for the complexation, the optimal ratio of PLPEI to DNA was determined based on the amounts of amine required to completely inhibit DNA migration on an agarose gel, since the complex formation hinders the migration of DNA, retaining the DNA in the wells. Constant amounts of plasmid DNA were mixed with PLPEI at varying amine/phosphate (N/P) ratios and analyzed by agarose gel electrophoresis. The bound fraction of DNA was increased as the N/P ratio increased and the most DNA was bound at an N/P ratio higher than 6. The complexation profile of PLPEI was comparable to that of the unmodified PEI, indicating that the PEI capacity for DNA complexation was not diminished by lipid conjugation (FIG. 2a). An N/P ratio of 10, where all DNA is bound to the complexes, was chosen and used for the following steps.

For enveloping the PLPEI/DNA complexes, a mixture of free lipids comprising POPC, cholesterol, PEG-PE (3:3:0.3 mol/mol) was separately prepared as an aqueous suspension. The lipid suspension was then incubated with the preformed PLPEI/DNA complexes leading to spontaneous envelope formation, most probably a monolayer, driven by hydrophobic interaction between the lipid moieties of PLPEI and free lipids (post-insertion technique). The optimal amounts of the free lipid were estimated approximately from the number of lipid molecules that would provide a complete monolayer envelope to the preformed PLPEI/DNA complexes. It was calculated, assuming that a bilayer liposome with 50 nm diameter contains about 25,000 lipid molecules[18, 19] and PLPEI/DNA cores have a mass/volume ratio of 1 g/ml, that about 0.2 μmole of total lipids is required to cover the entire surface of the particulate cores with diameters of 50 nm and a total mass of 230 μg, i.e., one μmole of total lipids is required to cover completely the surface of the particulate cores with one milligram of the total mass. Thus, unless otherwise mentioned, 100 μg of DNA was complexed with 180 μg of PLPEI corresponding 131 μg (0.08 μmole) of PEI and 49 μg (0.08 μmole) of PL and then incubated with 42 μg (0.055 μmole) POPC, 21 μg (0.055 μmole) cholesterol and 15 μg (0.005 μmole) PEG-PE.

The interaction and incorporation of the free lipids into the PLPEI/DNA complexes was confirmed by the colocalization of the fluorescently-labeled free lipid (CF-PEG-PE) with the fluorescently-labeled DNA (Rh-DNA) under the fluorescence microscopy (not shown). The characteristic hard-core structure with monolayer envelope was clearly confirmed by the freeze-fracture transmission electron microscopy (ffTEM). ffTEM revealed well-developed spherical nanoparticles with a mean diameter of 50 nm (FIG. 2b). All particles displayed their shadow behind the structures which is typical for “hard-core” particles including micelles. This behavior is different from the fracture behavior of bilayer-structures such as liposomes which display concave and convex fracture planes (shadow in front and behind the structure, respectively).

### Example II

### Physicochemical Properties of MNP

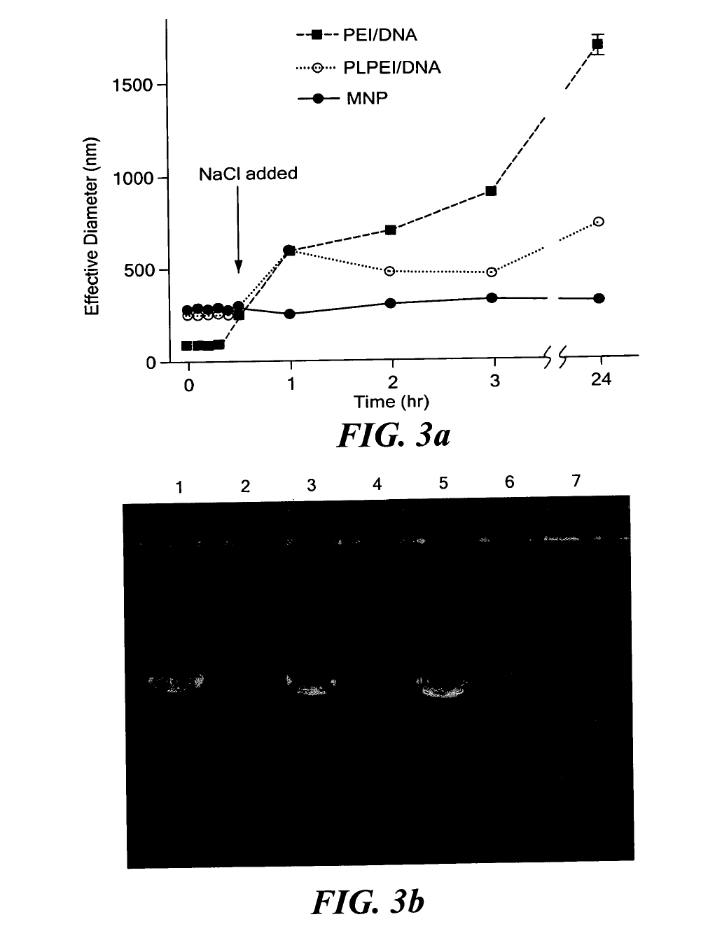

Traditional PEI/DNA polyplexes tend to aggregate rapidly under physiological high salt conditions [8]. To demonstrate the stabilizing effect of the lipid envelope against the salt-induced aggregation, NaCl was added to complex formulations to a final concentration of 0.15M while monitoring the hydrodynamic diameter. As expected, PEI/DNA polyplexes aggregated immediately after adding NaCl with continuous increases in hydrodynamic diameter up to almost 20-folds over a 24 hour period. The intermediate PLPEI/DNA complexes without free lipids and PEG-PE showed a two-fold increase immediately after adding NaCl and then remained relatively constant over the 24 hours. At the same time, MNP remained stable with no significant aggregation upon salt addition for 24 hours (FIG. 3a).

Zeta potential measurement revealed that MNP have a favorable neutral surface charge of −2.1±0.86 mV (mean±s.e.m., n=5), while PEI/DNA polyplexes have a more toxic positive surface charge of 20.2±1.38 mV (mean±s.e.m., n=5). The neutral surface charge of MNP also suggested the presence of the lipid layer which provided charge shielding of the otherwise positive PEI/DNA core.

The presence of the lipid layer was further demonstrated by the complete protection of the loaded DNA against the enzymatic degradation. The free DNA was completely degraded by the enzyme treatment while the DNA in either PEI/DNA or MNP remains intact. Migration of intact DNA was slightly retarded after enzyme treatment probably due to interference with the enzyme. Quantitation of intact DNA (ImageJ, NIH) revealed that 93% of loaded DNA was recovered from MNP as compared to only 70% recovery from PEI/DNA, supporting the notion of complete encapsulation of DNA within the lipid membrane (FIG. 3b).

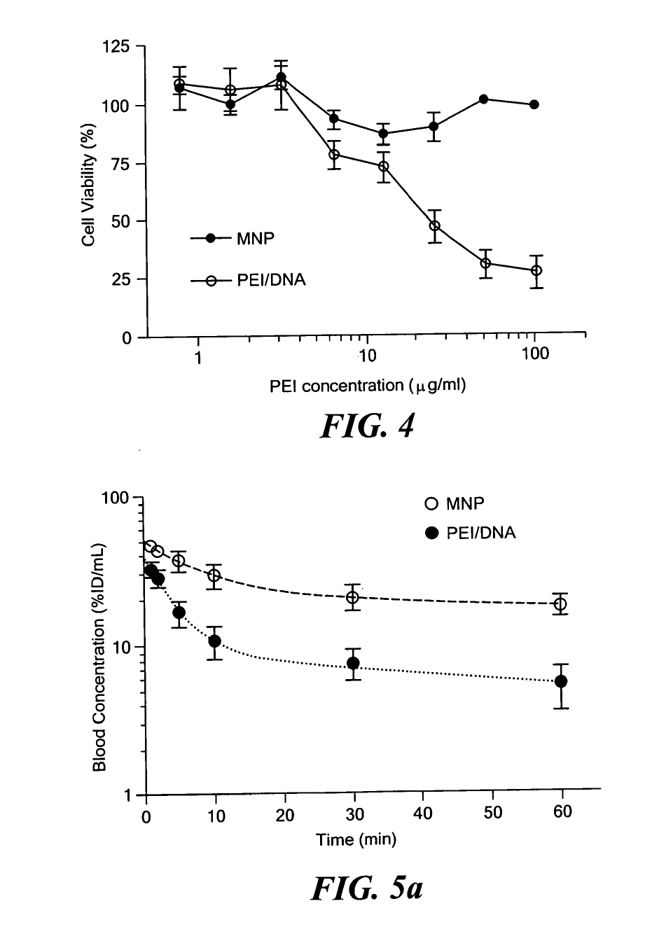

We have also evaluated the cytotoxicity of MNP towards the NIH/3T3 cells. MNP showed no toxicity at a PEI concentration of 100 μg/ml after 24 hrs of incubation that followed 4 hrs of treatment in striking contrast with PEI/DNA complexes, which were highly toxic at a PEI concentration of 15 μg/ml (FIG. 4). This result looks quite understandable in light of the data showing a neutral surface charge on MNP compared to the strong positive charge on the surface of PEI/DNA complexes.

### Example III

### In Vivo Biodistribution and Gene Expression

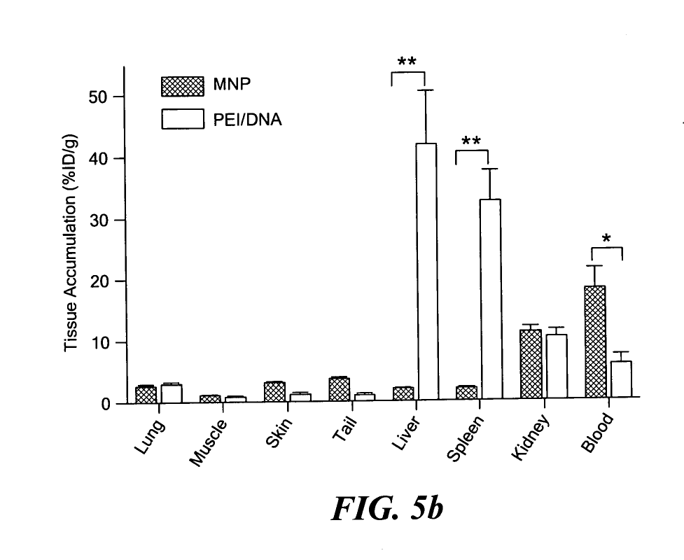

To demonstrate the prolonged circulation time of MNP in the blood and thus the feasibility of their enhanced delivery to target tissues such as tumors, pharmacokinetic and biodistribution studies were performed with MNP loaded with ¹¹¹In-DNA in mice. The radioactivity in major organs after i.v. bolus administration of MNP loaded with ¹¹¹In-DNA was measured and compared to that of control PEI/¹¹¹In-DNA complexes. After 10 min, as much as 30% ID/ml of MNP remained in the blood compared to about 10% ID/ml for PEI/DNA polyplexes. At 1 hour post-injection, about 20% ID/ml of MNP was still present in the blood, while only about 5% ID/ml of PEI/DNA polyplexes was detected in the circulation (FIG. 5a).

The slower clearance and thus more prolonged circulation of DNA in MNP compared to PEI/DNA were also confirmed by pharmacokinetic parameters. The half-life (t_{1/2 beta}) was estimated by fitting the blood concentration data colleted to 60 minutes to a two-compartment model and found to be approximately 239 minutes as compared to 33 minutes for PEI/DNA polyplexes. The area under the curve (AUC) obtained from the “concentration vs time” curves also revealed a significant increase in the systemic availability of plasmid DNA in MNP compared to the polyplexes of PEI (1404% ID·min/ml vs. 530% ID·min/ml). The extended circulation time was due to the reduced clearance by the RES uptake. While the DNA in the control polyplexes accumulated mainly in RES organs (40% ID/g liver and 30% ID/g spleen), the DNA in MNP bypassed the RES organs with significantly reduced accumulation (less than 5% ID/g for liver and spleen) (FIG. 5b). Taken together, the long circulation time along with low accumulation in RES sites makes MNP suitable for in vivo application.

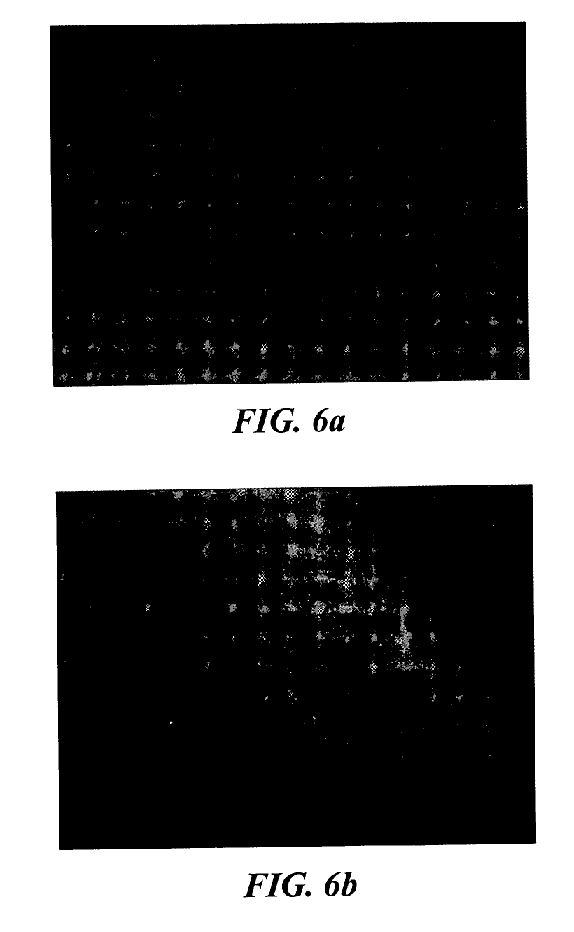

The feasibility of the enhanced gene delivery and in vivo transfection of targets, such as tumors, suitable for the enhanced permeability and retention (EPR) effect-mediated passive accumulation of long-circulating pharmaceutical nanocarriers was demonstrated in mice bearing the LLC tumor. Gene expression at the tumor tissue was accessed following the i.v. administration of MNP loaded with the plasmid DNA encoding for the Green Fluorescence Protein (GFP). At 48 hours post-injection, bright GFP fluorescence was observed in tumors from the animals treated with MNP whereas no fluorescence was found in tumors from the control mice (FIG. 6). GFP expression in tumor tissues from the animals injected with PEI/DNA polyplexes was not accessed due to short survival of the animals. The intravenous administration of PEI/DNA polyplexes at a comparable dose caused death of the animals within 30 min from respiratory failure, additionally confirming significantly decreased toxicity of MNP.

Taken together, the prolonged circulation in the blood along with a low accumulation in RES sites allowed for a significant accumulation of MNP at the tumor site, leading to strong reporter gene expression. These qualities of MNP make them suitable for the in vivo gene therapy.

### Example IV

### Preparation of siRNA-Loaded MNP

For the preparation of siRNA-loaded MNP, similar to DNA, siRNA is first complexed with PLPEI at the same N/P ratio of 10 as for the preparation of DNA-containing MNP. A chosen quantity of siRNA is mixed with PLPEI used in the required quantity to provide an N/P ratio of 10. Note that an equal quantity of antisense oligonucleotide could be substituted for the siRNA in order to prepare antisense-loaded MNP. The siRNA/PLPEI complexes so formed are used for the following steps.

Separately, a mixture of free lipids including POPC, cholesterol, PEG2000-DSPE (3:3:0.3 mol/mol) is prepared as an aqueous suspension. The free lipid suspension is then incubated with the preformed PLPEI/DNA complexes. Assuming siRNA/PEI cores have a mass/volume ratio of 1 g/ml, about 0.2 μmole of total lipids is required to cover all the surface of the particulate cores with diameters of 50 nm and a total mass of 230 μg; i.e., one μmole of total lipids is required to cover the entire surface of the particulate cores with one milligram of total mass.

The amount of PEG-PE, i.e., 10 mol % of free and 4.3 mol % of total phospholipids, is chosen to facilitate incorporation of free lipids into the preformed complexes and also to provide steric stabilization to the final construct. Upon incubation with the preformed PLPEI/DNA complexes, the PEG-PE content of total lipids comprising the free and the conjugated lipids decreases to 4.3 mol %, at which a lamellar phase is favored. The final construct is a sterically stabilized micelle-like hard-core particle with an siRNA/PEI polyplex core and lipid monolayer envelope.

The interaction and incorporation of the free lipids into the siRNA/PLPEI complexes is confirmed by co-localization of fluorescent-labeled free lipid (CF-PEG2000-DSPE) with fluorescent-labeled siRNA (Cy5-siRNA) using fluorescence microscopy. The characteristic hard-core structure with monolayer envelope is confirmed by freeze-fracture transmission electron microscopy (ffTEM).

### REFERENCES

[1] O. Boussif, F. Lezoualc'h, M. A. Zanta, M. D. Mergny, D. Scherman, B. Demeneix, J. P. Behr, A versatile vector for gene and oligonucleotide transfer into cells in culture and in vivo: polyethylenimine. Proc Natl Acad Sci USA 92(16) (1995) 7297-7301.
[2] A. Akinc, M. Thomas, A. M. Klibanov, R. Langer, Exploring polyethylenimine-mediated DNA transfection and the proton sponge hypothesis. J Gene Med 7(5) (2005) 657-663.
[3] R. Kircheis, L. Wightman, E. Wagner, Design and gene delivery activity of modified polyethylenimines. Adv Drug Deliv Rev 53(3) (2001) 341-358.
[4] S. V. Vinogradov, E. V. Batrakova, A. V. Kabanov, Nanogels for oligonucleotide delivery to the brain. Bioconjug Chem 15(1) (2004) 50-60.
[5] A. Kichler, Gene transfer with modified polyethylenimines. J Gene Med 6 Suppl 1 (2004) S3-10.
[6] S. Brunner, E. Furtbauer, T. Sauer, M. Kursa, E. Wagner, Overcoming the nuclear barrier: cell cycle independent nonviral gene transfer with linear polyethylenimine or electroporation. Mol Ther 5(1) (2002) 80-86.
[7] T. Merdan, J. Kopecek, T. Kissel, Prospects for cationic polymers in gene and oligonucleotide therapy against cancer. Adv Drug Deliv Rev 54(5) (2002) 715-758.
[8] M. Neu, D. Fischer, T. Kissel, Recent advances in rational gene transfer vector design based on poly(ethylene imine) and its derivatives. J Gene Med 7(8) (2005) 992-1009.
[9] D. Oupicky, M. Ogris, L. W. Seymour, Development of long-circulating polyelectrolyte complexes for systemic delivery of genes. J Drug Target 10(2) (2002) 93-98.
[10] S. M. Moghimi, A. C. Hunter, J. C. Murray, Long-circulating and target-specific nanoparticles: theory to practice. Pharmacol Rev 53(2) (2001) 283-318.
[11] M. Ogris, S. Brunner, S. Schuller, R. Kircheis, E. Wagner, PEGylated DNA/transferrin-PEI complexes: reduced interaction with blood components, extended circulation in blood and potential for systemic gene delivery. Gene Ther 6(4) (1999) 595-605.
[12] H. Petersen, P. M. Fechner, A. L. Martin, K. Kunath, S. Stolnik, C. J. Roberts, D. Fischer, M. C. Davies, T. Kissel, Polyethylenimine-graft-poly(ethylene glycol) copolymers: influence of copolymer block structure on DNA complexation and biological activities as gene delivery system. Bioconjug Chem 13(4) (2002) 845-854.
[13] D. Finsinger, J. S. Remy, P. Erbacher, C. Koch, C. Plank, Protective copolymers for nonviral gene vectors: synthesis, vector characterization and application in gene delivery. Gene Ther 7(14) (2000) 1183-1192.
[14] Y. Yamazaki, M. Nango, M. Matsuura, Y. Hasegawa, M. Hasegawa, N. Oku, Polycation liposomes, a novel nonviral gene transfer system, constructed from cetylated polyethylenimine. Gene Ther 7(13) (2000) 1148-1155.
[15] D. A. Wang, A. S. Narang, M. Kotb, A. O. Gaber, D. D. Miller, S. W. Kim, R. I. Mahato, Novel branched poly(ethylenimine)-cholesterol water-soluble lipopolymers for gene delivery. Biomacromolecules 3(6) (2002) 1197-1207.
[16] J. Heyes, L. Palmer, K. Chan, C. Giesbrecht, L. Jeffs, I. MacLachlan, Lipid encapsulation enables the effective systemic delivery of polyplex plasmid DNA. Mol Ther 15(4) (2007) 713-720.
[17] M. Nishikawa, T. Nakano, T. Okabe, N. Hamaguchi, Y. Yamasaki, Y. Takakura, F. Yamashita, M. Hashida, Residualizing indium-111-radiolabel for plasmid DNA and its application to tissue distribution study. Bioconjug Chem 14(5) (2003) 955-961.
[18] F. J. Hutchinson, S. E. Francis, I. G. Lyle, M. N. Jones, The characterisation of liposomes with covalently attached proteins. Biochim Biophys Acta 978(1) (1989) 17-24.
[19] C. B. Hansen, G. Y. Kao, E. H. Moase, S. Zalipsky, T. M. Allen, Attachment of antibodies to sterically stabilized liposomes: evaluation, comparison and optimization of coupling procedures. Biochimica et biophysica Acta 1239(2) (1995) 133-144.
[20] M. C. Woodle, D. D. Lasic, Sterically stabilized liposomes. Biochim Biophys Acta 1113(2) (1992) 171-199.
[21] T. Reschel, C. Konak, D. Oupicky, L. W. Seymour, K. Ulbrich, Physical properties and in vitro transfection efficiency of gene delivery vectors based on complexes of DNA with synthetic polycations. J Control Release 81(1-2) (2002) 201-217.
[22] D. Fischer, B. Osburg, H. Petersen, T. Kissel, U. Bickel, Effect of poly(ethylene imine) molecular weight and pegylation on organ distribution and pharmacokinetics of polyplexes with oligodeoxynucleotides in mice. Drug Metab Dispos 32(9) (2004) 983-992.
[23] K. Kunath, A. von Harpe, H. Petersen, D. Fischer, K. Voigt, T. Kissel, U. Bickel, The structure of PEG-modified poly(ethylene imines) influences biodistribution and pharmacokinetics of their complexes with NF-kappaB decoy in mice. Pharm Res 19(6) (2002) 810-817.
[24] M. Neu, O. Germershaus, M. Behe, T. Kissel, Bioreversibly crosslinked polyplexes of PEI and high molecular weight PEG show extended circulation times in vivo. J Control Release 124(1-2) (2007) 69-80.
[25] M. Johnsson, K. Edwards, Liposomes, disks, and spherical micelles: aggregate structure in mixtures of gel phase phosphatidylcholines and poly(ethylene glycol)-phospholipids. Biophys J 85(6) (2003) 3839-3847.
[26] G. Montesano, R. Bartucci, S. Belsito, D. Marsh, L. Sportelli, Lipid membrane expansion and micelle formation by polymer-grafted lipids: scaling with polymer length studied by spin-label electron spin resonance. Biophys J 80(3) (2001) 1372-1383.
[27] K. Sou, T. Endo, S. Takeoka, E. Tsuchida, Poly(ethylene glycol)-modification of the phospholipid vesicles by using the spontaneous incorporation of poly(ethylene glycol)-lipid into the vesicles. Bioconjug Chem 11(3) (2000) 372-379.
[28] C. L. Gebhart, S. Sriadibhatla, S. Vinogradov, P. Lemieux, V. Alakhov, A. V. Kabanov, Design and formulation of polyplexes based on pluronic-polyethyleneimine conjugates for gene transfer. Bioconjug Chem 13(5) (2002) 937-944.
[29] A. V. Kabanov, E. V. Batrakova, V. Y. Alakhov, Pluronic block copolymers as novel polymer therapeutics for drug and gene delivery. J Control Release 82(2-3) (2002) 189-212.
[30] R. J. Lee, L. Huang, Folate-targeted, Anionic Liposome-entrapped Polylysine-condensed DNA for Tumor Cell-specific Gene Transfer. Journal of Biological Chemistry 271(14) (1996) 8481-8487.
[31] K. Muller, T. Nande, A. Fahr, R. Muller, S. Brusselbach, Highly efficient transduction of endothelial cells by targeted artificial virus-like particles. Cancer Gene Therapy 8(2) (2001) 107-117.
[32] P. Bandyopadhyay, X. Ma, C. L. Stieers, B. T. Kren, C. J. Steer, Nucleotide Exchange in Genomic DNA of Rat Hepatocytes Using RNA/DNA Oligonucleotides. Journal of Biological Chemistry 274(15) (1999) 10163-10172.
[33] J. J. Wheeler, L. Palmer, M. Ossanlou, I. MacLachlan, R. W. Graham, Y. P. Zhang, M. J. Hope, P. Scherrer, P. R. Cullis, Stabilized plasmid-lipid particles: construction and characterization. Gene Ther 6(2) (1999) 271-281.
[34] I. MacLachlan, P. Cullis, R. W. Graham, Progress towards a synthetic virus for systemic gene therapy. Curr Opin Mol Ther 1(2) (1999) 252-259.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. A nanoparticle comprising a core complex encapsulated by a lipid monolayer, wherein the core complex comprises one or more nucleic acid molecules electrostatically bound to one or more molecules of a cationic polymer, wherein the cationic polymer is covalently conjugated to a first lipid residing in the lipid monolayer.

2. The nanoparticle of claim 1, wherein the cationic polymer comprises linear or branched polyethyleneimine, polyornithine, polyarginine, polylysine, polyallylamine, aminodextran, or any combination thereof.

3. The nanoparticle of claim 1, wherein the first lipid is selected from the group consisting of natural or synthetic phospholipids, glycolipids, aminolipids, sphingolipids, long chain fatty acids, and sterols.

4. The nanoparticle of claim 1, wherein the lipid monolayer further comprises one or more non-conjugated lipids.

5. The nanoparticle of claim 4, wherein the one or more non-conjugated phospholipid molecules are selected from the group consisting of natural or synthetic phospholipids, glycolipids, aminolipids, sphingolipids, long chain fatty acids, and sterols.

6. The nanoparticle of claim 4, wherein a portion of the non-conjugated phospholipid molecules are PEGylated.

7. The nanoparticle of claim 6, wherein the lipid monolayer comprises PEG-phosphatidylethanolamine or pNP-PEG-PE.

8. The nanoparticle of claim 1, wherein the lipid monolayer further comprises cholesterol.

9. The nanoparticle of claim 8, wherein the lipid monolayer comprises conjugated first lipid, non-conjugated lipid, and cholesterol at a molar ratio of 4:3:3.

10. The nanoparticle of claim 8 further comprising PEG-phosphatidylethanolamine, wherein the lipid monolayer comprises conjugated first lipid, non-conjugated lipid, cholesterol, and PEG-phosphatidylethanolamine at a molar ratio of 4:3:3:0.3.

11. The nanoparticle of claim 1, wherein the one or more nucleic acid molecules comprise an oligonucleotide, a DNA molecule, an RNA molecule, or any combination thereof.

12. The nanoparticle of claim 11, wherein the one or more nucleic acid molecules comprise plasmid DNA, RNAi, siRNA, an antisense oligonucleotide, or a ribozyme.

13. The nanoparticle of claim 11, wherein the one or more nucleic acid molecules comprise a therapeutic gene.

14. The nanoparticle of claim 13, wherein the therapeutic gene is a cytotoxic or suicide gene.

15. The nanoparticle of claim 1, wherein the one or more nucleic acid molecules comprise up to 40% by weight of the particle.

16. The nanoparticle of claim 15, wherein the one or more nucleic acid molecules comprise about 25% by weight of the particle.

17. The nanoparticle of claim 1, wherein the cationic polymer is covalently bound to a distal end of an alkyl or acyl chain of the first lipid.

18. The nanoparticle of claim 1, wherein the diameter of the particle is about 50 nm.

19. A non-viral vector comprising the nanoparticle of claim 1.

20. The vector of claim 19 further comprising a targeting agent.

21. The vector of claim 20, wherein the targeting agent is selected from the group consisting of an antibody or antigen-binding fragment thereof, a single-chain antibody, a domain antibody, a ligand for a cell-surface receptor, and biotin.

22. The vector of claim 21, wherein the targeting agent is coupled to the vector by a cleavable bond.

23. The vector of claim 22, wherein the cleavable bond is cleaved at low pH.

24. The vector of claim 23, wherein the cleavable bond is a hydrazone bond.

25. The vector of claim 22, wherein the cleavable bond is the bond coupling the cationic polymer to the first lipid molecule.

26. A method of making a nanoparticle according to claim 1, the nanoparticle comprising a core complex encapsulated by a lipid monolayer, the method comprising:
(a) providing a nucleic acid, a cationic polymer-lipid covalent conjugate, and one or more non-conjugated lipids;
(b) contacting the nucleic acid and the cationic polymer-lipid conjugate under conditions suitable to form the core complex, the core complex comprising the nucleic acid electrostatically bound to the cationic polymer portion of the conjugate; and
(c) contacting the core complex and the non-conjugated lipid to form the lipid monolayer.

27. The method of claim 26, wherein the nucleic acid and cationic polymer-lipid conjugate are contacted in step (b) in solution to form the core complex.

28. The method of claim 26, wherein the non-conjugated lipid is provided in the form of a dry film, and the dry film is hydrated prior to performing step (c).

29. The method of claim 27, wherein the non-conjugated lipid is provided in the form of a dry film and the aqueous suspension of core complex from step (b) is used to hydrate the dry film during step (c).

30. The method of claim 26, further comprising adding to the non-conjugated lipid prior to step (c) a component selected from the group consisting of a neutral lipid, a glycolipid, a PEGylated lipid, a biotinylated lipid, an acylated protein or glycoprotein, a protein or glycoprotein conjugated to a lipid, an antibody or antigen-binding fragment thereof, a single chain antibody, a domain antibody, and a ligand for a cell surface receptor.

31. The method of claim 30, wherein a neutral lipid is added, and the neutral lipid is cholesterol.

32. The method of claim 30, wherein a PEGylated lipid is added, and the PEGylated lipid is PEG-phosphatidylethanolamine or pNP-PEG-PE.

33. The method of claim 32 wherein a neutral lipid is added, the neutral lipid is cholesterol, and the molar ratio of the polymer-lipid conjugate, non-conjugated lipid, cholesterol, and PEG-phosphatidylethanolamine is 4:3:3:0.3.

34. A method of transfecting a cell, the method comprising contacting the cell with a non-viral vector according to claim 19, wherein a nucleic acid molecule of the vector is transferred into the cell.

35. A method of suppressing the expression of a gene in a cell, the method comprising contacting the cell with a nanoparticle according to claim 1, wherein the nanoparticle comprises siRNA or RNAi, and wherein the siRNA or RNAi is transferred into the cell and suppresses the expression of the gene.

36. A method of treating a subject having a disease or medical condition, the method comprising administering to the subject a non-viral vector according to claim 19, wherein a nucleic acid molecule of the vector is transferred into cells of the subject, whereby the disease or medical condition is treated.

37. The method of claim 36, wherein the disease is cancer.

38. The method of claim 37, wherein the vector is targeted to a tumor.

39. A chemical conjugate comprising a cationic polymer covalently bound to a distal end of a lipid acyl or alkyl chain.

40. The chemical conjugate of claim 39 comprising polyethyleneimine.

41. The chemical conjugate of claim 40 formed by reacting 1-palmitoyl-2-azelaoyl-sn-glycero-3-phosphocholine with branched polyethyleneimine.

42. A complex of the chemical conjugate of claim 39 and a nucleic acid.

43. A micelle, lipid monolayer, or lipid bilayer structure comprising the conjugate of claim 39.
